SEQUENCE LISTING (6 sequences)

SEQ ID NO: 1 - DNA, 22 bp, Artificial Sequence
  synthetic antisense phosphorodiamidate morpholino oligomer
ctgctgctgctgctgctgctgc

SEQ ID NO: 2 - DNA, 25 bp, Artificial Sequence
  synthetic antisense phosphorodiamidate morpholino oligomer
ctgctgctgctgctgctgctgctgc

SEQ ID NO: 3 - DNA, 28 bp, Artificial Sequence
  synthetic antisense phosphorodiamidate morpholino oligomer
ctgctgctgctgctgctgctgctgctgc

SEQ ID NO: 4 - DNA, 25 bp, Artificial Sequence
  synthetic antisense phosphorodiamidate morpholino oligomer
ccttcatcagcttttccagggtcgc

SEQ ID NO: 5 - DNA, 25 bp, Artificial Sequence
  synthetic antisense phosphorodiamidate morpholino oligomer
gctgctgctgctggaaggacttgag

SEQ ID NO: 6 - DNA, 25 bp, Artificial Sequence
  synthetic antisense phosphorodiamidate morpholino oligomer
cctcttacctcagttacaatttata

(19) United States
(12) Patent Application Publication
(10) Pub. No.: US 2016/0017327 A1
(43) Pub. Date: Jan. 21, 2016

(54) PHOSPHORODIAMIDATE MORPHOLINO OLIGOMERS (PMOS) AND THEIR USE IN SUPPRESSION OF MUTANT HUNTINGTIN EXPRESSION AND ATTENUATION OF NEUROTOXICITY

(71) Applicant: THE JOHNS HOPKINS UNIVERSITY, BALTIMORE, MD (US)

(72) Inventors: Dobrila D. Rudnicki, Baltimore, MD (US); Russell L. Margolis, Baltimore, MD (US)

(21) Appl. No.: 14/791,510

(22) Filed: Jul. 6, 2015

Related U.S. Application Data
(60) Provisional application No. 62/023,334, filed on Jul. 11, 2014.

(51) Int. Cl. C12N 15/113; A61K 31/7125

(52) U.S. Cl. CPC C12N 15/113; A61K 31/7125; C12N 2310/11; C12N 2310/3233; C12N 2310/31; C12N 2320/30

(57) ABSTRACT

The present invention provides antisense phosphorodiamidate morpholino oligomers which are useful for the suppression or inhibition of the HTT gene involved in Huntington's disease. The oligomers can selectively suppress mutant forms of the HTT protein while allowing the normal protein to be expressed in sufficient quantity to retain its function in the cell. Methods for treatment of Huntington's disease are also provided.

### REFERENCE TO RELATED APPLICATIONS

This application claims the benefit of U.S. Provisional Patent Application No. 62/023,334, filed on Jul. 11, 2014, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Jul. 9, 2014, is named P12049_ST25.txt and is 1,701 bytes in size.

### BACKGROUND OF THE INVENTION

The expansion of CAG trinucleotide repeats leads to at least nine autosomal dominant neurodegenerative diseases. Among them, Huntington's disease (HD), characterized by progressive motor, cognitive, and psychiatric abnormalities leading to death 15-20 years after clinical onset is the most common HD is caused by an expansion of the CAG repeat in the first exon of the HTT gene; disease inevitably results in individuals with 40 or more triplets, and may occur with as few as 36 triplets (and perhaps fewer). The CAG repeat is translated into a polyglutamine tract (polyQ) within the huntingtin protein (HTT). Most investigators have concluded that toxicity of the expanded polyglutamine tract is the primary pathogenic mechanism in HD. Recently, it was shown that CAG repeats, including at the HD locus, can be translated into other homopolymeric tracts, including polyalanine and polyserine, through repeat-associated non-ATG (RAN) translation. In addition, mutant HTT RNA itself may be toxic, suggesting that both RNA and protein gain-of-function contribute to the disease pathogenesis.

If both expanded HTT protein and transcript are neurotoxic, then the most direct therapeutic approach, aside from altering genomic DNA, is to use knockdown strategies to prevent protein expression and degrade expanded transcripts or block their toxicity. While ideally suppression is specific for the products of the expanded allele, the current consensus is that bi-allelic approaches may be successful as long as the level of normal HTT remains above the 30% threshold required for normal cell function. Multiple strategies are under investigation, such as zinc finger peptides target double-stranded DNA to prevent transcription, therefore reducing either protein and RNA expression, or peptides that bind to expanded polyglutamine to block its toxic function. However, most mutant HTT knockdown strategies are based on small interfering RNAs (siRNAs) and antisense oligonucleotides (ASOs). Published evidence demonstrates the potential of these approaches to significantly decrease the expression of expanded HTT protein without completely blocking expression of normal HTT, or at least to preserve a minimally needed amount of normal HTT. One strategy takes advantage of the heterozygosity of single-nucleotide polymorphisms (SNPs) in HTT; the mutant HTT gene contains specific SNPs in 75-85% of HD patients, and targeting these SNPs with one of a pool of siRNAs or ASOs could provide allele-specific knockdown of HTT expression.

A second approach to allelic specificity employs siRNAs or ASOs that target the expanded CAG repeat. Indeed, CAG repeat-targeting siRNAs are effective at reducing the expression of mutant HTT mRNA with at least partial allelic selectivity in vitro. However, implementation of siRNA-based silencing in vivo faces several major obstacles, including the challenge of efficient delivery into the CNS, the relatively low stability of siRNAs, potential off-target effects, and the risk of activation of the immune system.

Compared to siRNA, ASOs have a major advantage in flexibility, as modifications can enhance their stability, RNA affinity, cellular uptake and biodistribution. ASOs such as gapmers [chimeric ASOs consisting of a DNA sequence with flanking locked nucleic acids (LNA) or 2′-O-Methoxyethyl (MOE) nucleic acids] can be used for RNase H-dependent degradation of targeted transcripts, an approach used to degrade CUG repeats in mouse models. However, a number of CAG repeat loci, including the HD locus, contain antisense transcripts spanning the repeat region (38-40), and it is possible that targeted degradation of sense transcripts may trigger an upregulation of CUG repeat-containing antisense transcripts with potential neurotoxic effects. An alternative is to use ASOs that sterically block RNA translation and potentially RNA-mediated neurotoxicity without leading to transcript degradation. Such ASOs include peptide nucleic acids (PNAs), LNAs, chemically modified single-stranded RNAs (ssRNAs), and phosphorodiamidate morpholino oligonucleotides (PMOs).

There still exists an unmet need for novel strategies that selectively reduce mutant HTT expression in patients with HD, without loss of normal HTT function and off target effects.

### SUMMARY OF THE INVENTION

In accordance with one or more embodiments, the present inventors designed multiple novel PMOs targeted to the HTT CAG repeat region and to the region flanking the HTT repeat. The effectiveness and allelic selectivity of the PMOs were examined in HD-patient derived fibroblast lines with repeat expansions of 44, 77 or 109 CAG triplets and in two HD mouse models, N171-82Q transgenic and Hdh^{Q7/Q150} knock-in mice. The effect on ameliorating neurotoxicity was examined in a neuroblastoma cell line. The PMOs of the present invention described herein can significantly decrease HTT protein expression without decreasing transcript level, with target selectivity and knock-down efficacy determined by PMO sequence and concentration, and by target CAG repeat length.

In accordance with an embodiment, the present invention provides an antisense phosphorodiamidate morpholino oligomer having the formula:

5′-(CTG)ₙC-3 (I),

wherein n=5 to 15 trinucleotide repeats.

In accordance with another embodiment, the present invention provides an antisense phosphorodiamidate morpholino oligomer, wherein the antisense phosphorodiamidate morpholino oligomer is selected from the group consisting of:

(CTG22)
(SEQ ID NO: 1)
5′-CTGCTGCTGCTGCTGCTGCTGC-3′,
(CTG25)
(SEQ ID NO: 2)
5′-CTGCTGCTGCTGCTGCTGCTGCTGC-3′,
and
(CTG28)
(SEQ ID NO: 3)
5′-CTGCTGCTGCTGCTGCTGCTGCTGCTGC-3′.

In accordance with an embodiment, the present invention provides an antisense phosphorodiamidate morpholino oligomer having a polynucleotide sequence of between 20 to 30 nucleotides in length, which is complimentary to the nucleotides immediately 3′ to the start codon of the HTT gene mRNA.

In accordance with a further embodiment, the present invention provides an antisense phosphorodiamidate morpholino oligomer having the sequence:

(HTTex1a)
(SEQ ID NO: 4)
5′-CCTTCATCAGCTTTTCCAGGGTCGC-3′.

In accordance with an embodiment, the present invention provides an antisense phosphorodiamidate morpholino oligomer having a polynucleotide sequence of between 20 to 30 nucleotides in length, which is complimentary to the nucleotides immediately 5′ from the start of the CAG repeat region of the HTT gene mRNA.

In accordance with another embodiment, the present invention provides an antisense phosphorodiamidate morpholino oligomer having the sequence:

(HTTex1b)
(SEQ ID NO: 5)
5′-GCTGCTGCTGCTGGAAGGACTTGAG-3′.

In accordance with an embodiment, the present invention provides a composition comprising at least one or more of the antisense phosphorodiamidate morpholino oligomers described herein.

In accordance with another embodiment, the present invention provides a pharmaceutical composition comprising at least one or more of the antisense phosphorodiamidate morpholino oligomers described herein, and a pharmaceutically acceptable carrier.

In accordance with a further embodiment, the present invention provides a pharmaceutical composition comprising at least one or more of the antisense phosphorodiamidate morpholino oligomers described herein, at least one additional biologically active agent, and a pharmaceutically acceptable carrier.

In accordance with another embodiment, the present invention provides a method for reducing or inhibiting expression of HTT protein in a cell comprising contacting the cell with at least one or more of the antisense phosphorodiamidate morpholino oligomers described herein.

In accordance with still another embodiment the present invention provides a method for reducing or inhibiting expression of HTT protein in a subject comprising administering to the subject an effective amount of the pharmaceutical compositions described herein.

In accordance with another embodiment, the present invention provides a method for treatment of Huntington's disease in a subject comprising administering to the subject an effective amount of a pharmaceutical composition comprising at least one or more of the antisense phosphorodiamidate morpholino oligomers described herein.

### BRIEF DESCRIPTION OF THE DRAWINGS

FIG. 1 is a schematic representation of the PMO sequences and their target regions within human HTT mRNA.

FIGS. 2A-2C depict PMO CTG25 inhibits HTT expression. Cell lines HD 18/44, HD 20/77 and HD 19/109 were each treated for 48 hours with 1, 5, 10, and 20 μM CTG25 and 20 μM standard control PMO (Ctrl) (5′-CCTCTTACCTCAGTTACAATTTATA-3′) (SEQ ID NO: 5). (A) Analysis of total HTT levels after CTG25 treatment. Antibody MAB2166 was used to discriminate between normal HTT (lower band) and mutant HTT (upper band) in cell lines HD 20/77 and HD 19/109. Since it is not possible to resolve normal and mutant HTT in the HD 18/44 cell line, total protein levels were analyzed. HD 18/44, one-way ANOVA, F=2.960, P=0.0747, n=3; HD 20/77, two-way ANOVA, F(Allele)=0.51, P(Allele)=0.4821, F(PMO)=63.07, P(PMO)<0.0001, n=3; HD 19/109, two-way ANOVA, F(Allele)=1.35, P(Allele)=0.2571, F(PMO)=4.38, P(PMO)=0.0057, n=3; post-hoc test *P<0.05, **P<0.01, ***P<0.001 versus Ctrl PMO-treated group. (B) In addition, 1C2 immunoblotting was performed to assess mutant HTT level only. All HD cell lines, one-way ANOVA, n=3. HD 18/44, F=5.237, P=0.0154; HD 20/77, F=21.56, P<0.0001; HD 19/109, F=5.210, P=0.0039; post-hoc test *P<0.05, **P<0.01, ***P<0.001 versus Ctrl PMO-treated group. (C) Representative western blot data. HTT antibodies are shown in parentheses. All experiments were performed three times.

FIGS. 3A-3C show that PMO CTG28 preferentially reduces mutant HTT expression. Cell lines HD 18/44, HD 20/77 and HD 19/109 were each treated with 1, 5, 10, and 20 μM CTG28 or 20 μM standard control PMO (Ctrl). (A) Analysis of normal versus expanded HTT level. HD 18/44, one-way ANOVA, F=1.015, P=0.4449, n=3; HD 20/77, two-way ANOVA, F(Allele)=1.11, P(Allele)=0.3053, F(PMO)=3.27, P(PMO)=0.0323, n=3; HD 19/109, two-way ANOVA, F(Allele)=9.56, P(Allele)=0.0058, F(PMO)=14.35, P(PMO)<0.0001, n=3; post-hoc test **P<0.01, ***P<0.001 versus Ctrl PMO-treated group; #P<0.05 versus normal HTT. (B) The levels of mutant HTT following CTG28 treatment. All HD cell lines, one-way ANOVA, n=3. HD 18/44, F=2.256, P=0.1356; HD 20/77, F=0.7091, P=0.6039; HD 19/109, F=9.021, P=0.0024; post-hoc test **P<0.01 versus Ctrl PMO-treated group. (C) Representative western blot data. HTT antibodies are shown in parentheses. All experiments were performed three times.

FIGS. 4A-4C depict concentration- and targeted CAG repeat length-dependent reduction of HTT expression following treatment with PMO CTG22. Cell lines HD 18/44, HD 20/77 and HD 19/109 were each treated with 1, 5, 10, and 20 μM CTG22 or 20 μM standard control PMO (Ctrl) for 48 hours. (A) Analysis of the normal versus expanded HTT level after CTG22 treatment. HD 18/44, one-way ANOVA, F=4.380, P=0.0265, n=3; HD 20/77, two-way ANOVA, F(Allele)=0.03, P(Allele)=0.8677, F(PMO)=5.51, P(PMO)=0.0037, n=3; HD 19/109, two-way ANOVA, F(Allele)=0.83, P(Allele)=0.3740, F(PMO)=20.04, P(PMO)<0.0001, n=3; post-hoc test *P<0.05, **P<0.01, ***P<0.001 versus Ctrl PMO-treated group. (B) Mutant HTT level after CTG22 treatment. All HD cell lines, one-way ANOVA, n=3. HD 18/44, F=5.637, P=0.0122; HD 20/77, F=8.637, P=0.0028; HD 19/109, F=6.499, P=0.0076; post-hoc test *P<0.05, **P<0.01 versus Ctrl PMO-treated group. (C) Representative western blot data. HTT antibodies are shown in parentheses. All experiments were performed three times.

FIGS. 5A-5D show PMO off-target effects. Cell lines HD 18/44, HD 20/77, HD 19/109 and SH-SYSY neuroblastoma cell line were each treated with PMOs CTG22, CTG25 and CTG28, and the expression of ATXN2 (A), ATXN3 (B), TBP (C), AR and RAI1 (D) were assessed 48 hours after treatment. (A) ATXN2 expression was not significantly decreased following all PMO treatments. (B) The concentration range of 5-20 μM CTG22 significantly reduced the levels of ATXN3 in cell lines HD 20/77 and HD 19/109. CTG25 significantly inhibited ATXN3 expression in line HD 19/109. PMO CTG28 did not affect ATXN3 expression in any of the HD lines tested. (C) A high concentration (20 μM) of repeat-targeting PMOs had no significant effect on the level of TBP in line HD 19/109. (D) AR and RAI expression in the SH-SYSY cell line were not decreased by a high concentration (20 μM) of repeat-targeting PMOs. All treatments, one-way ANOVA, n=3. ATXN3 in HD 20/77 with CTG22, F=3.505, P=0.0490; ATXN3 in HD 19/109 with CTG22, F=4.050, P=0.0331; ATXN3 in HD 19/109 with CTG25, F=29.15, P<0.0001; post-hoc test *P<0.05, **P<0.01, ***P<0.001 versus Ctrl PMO-treated group. All experiments were performed three times.

FIGS. 6A-6D depict non-CAG repeat-targeting PMOs inhibit HTT expression in a non-allele-specific manner and without off-target effect. Cell line HD 19/109 was treated with non-repeat-targeting PMOs HTTex1a and HTTex1b. (A) Analysis of normal versus mutant HTT level. PMO HTTex1a, two-way ANOVA, F(Allele)=1.35, P(Allele)=0.2593, F(PMO)=10.39, P(PMO)=0.0001, n=3; PMO HTTex1b, two-way ANOVA, F(Allele)=0.03, P(Allele)=0.8706, F(PMO)=12.34, P(PMO)<0.0001, n=3; post-hoc test *P<0.05, **P<0.01, ***P<0.001 versus Ctrl PMO-treated group. (B) Mutant HTT level after HTTex1a and HTTex1b treatment. Both PMOs, one-way ANOVA, n=3. PMO HTTex1a, F=2.573, P=0.1028; PMO HTTex1b, F=8.018, P=0.0037; post-hoc test **P<0.01 versus Ctrl PMO-treated group. (C) As expected, PMOs HTTex1a and HTTex1b did not have an effect on the expression of endogenous ATXN2 and ATXN3 control proteins. Both PMOs, one-way ANOVA, n=3. (D) Representative western blot data. HTT antibodies are shown in parentheses. All experiments were performed three times.

FIGS. 7A-7B show that PMOs exhibit no significant effect on the RNA levels of HTT, HTTAS_v1 and endogenous control genes. (A) Cell line HD 19/109 was treated with 20 μM Ctrl, CTG22, CTG25, CTG28, HTTex1aoff-target and HTTex1b PMO for 48 hours, and RNA levels of HTT and endogenous control genes (ATXN1, ATXN3, TBP and ATN1) were examined using TaqMan real-time PCR. All genes, one-way ANOVA, n=3. (B) Cell line HD 18/44 was treated with 20 μM Ctrl and CTG22 PMO for 48 hours, and RNA level of HTTAS_v1 was examined using TaqMan real-time PCR. Student's t-test, n=3. Experiment was performed three times in triplicate.

FIGS. 8A-8C illustrate that PMOs show minimal cell toxicity and can protect cells against mutant HTT-associated neurotoxicity. (A) HEK293 cells were treated with each of the PMOs at the indicated concentrations. With the exception of CTG22, which showed high concentration-dependent toxicity in HEK293 cells, other PMOs show no or minimal cytotoxicity even at the highest levels. All PMO concentrations, one-way ANOVA, n=4; post-hoc test *P<0.05, ***P<0.001 versus Ctrl PMO-treated group. (B) Mouse neuronal cell lines STHdh Q7/Q7 and STHdh Q111/Q111 were treated with the indicated concentrations of L-glutamate (L-Glu) for 72 hours and a caspase 3/7 assay was used to identify the best L-Glu concentration to induce mutant HTT-related cytotoxicity for the subsequent studies. Two-way ANOVA, F(polyQ)=112.50, P(polyQ)<0.0001, F(Concentration)=97.06, P(Concentration)<0.0001, n=4; post-hoc test **P<0.01, ***P<0.001 versus STHdh Q7/Q7. (C) 48-hour pre-treatment of PMO CTG25 protected STHdh Q111/Q111 cells from HTT neurotoxicity. Two-way ANOVA, F(polyQ)=103.35, P(polyQ)<0.0001, F(Treatment)=11.45, P(Treatment)<0.0001, n=4; post-hoc test ***P<0.001, NS=no significance versus STHdh Q7/Q7. All experiments were performed three times in quadruplicate with similar results.

FIG. 9 shows PMO intracellular diffusion is cell type-specific. HD fibroblast cell line 19/109 and wild-type mouse primary cortical neurons were treated with 20 μM fluorescein-labeled standard control PMO (Ctrl) for 48 hours. In the HD fibroblasts, the majority of PMO remains within endosomes. PMO transfected into cultured primary cortical neurons is released from endosomes and diffuses in the cytoplasm and nucleus. Green, fluorescein-labeled Ctrl PMO (left top & bottom); Red, Rab7 as an endosome marker (middle top & bottom); Blue, 4′,6-diamidino-2-phenylindole (DAPI) as a nucleus marker (right top & bottom). Scale bar, 20 μm.

FIGS. 10A-10E depict CTG25 and CTG28 preferentially inhibit mutant HTT expression in vivo. Intracerebroventricular (ICV) injection was used to deliver 100 μg of standard control PMO (Ctrl) or CTG25 PMO into the right lateral ventricle of six-week-old N171-82Q transgenic HD mice. Two weeks following the injection, the mice were sacrificed and 1C2 immunoblotting was used to assess the levels of HTT N171-82Q protein. (A) CTG25 PMO successfully reduced HTT N171-82Q expression to 35-40% that of the control without any significant effect on the levels of endogenous HTT, as well as on control proteins ATXN2, ATXN3, AR and TBP. (B) Student's t-test, n=3; *P<0.05, ***P<0.001 versus Ctrl PMO-injected group. The same ICV injections were also used to deliver Ctrl PMO or CTG28 PMO to the right lateral ventricle of six-month-old HdhQ7/Q150 knock-in HD mice. Totally, three 100-μg ICV injections were administered bimonthly. Two months following the last injection, HdhQ7/Q150 knock-in HD mice were first the assessed with tail suspension test and then sacrificed to examine total HTT levels via MAB2166 immunoblotting. (C) CTG28 preferentially inhibited mutant HTT (upper mouse HTT band) to 15-45% that of control in multiple brain regions (cortex, striatum and cerebellum), with minimal or no effect on normal HTT (lower mouse HTT band). Cortex, two-way ANOVA, F(Allele)=180.9, P(Allele)<0.001, F(PMO)=115.9, P(PMO)<0.001, n=4; Striatum, two-way ANOVA, F(Allele)=190.6, P(Allele)<0.001, F(PMO)=7.84, P(PMO)=0.0161, n=4; Cerebellum, two-way ANOVA, F(Allele)=155.2, P(Allele)<0.001, F(PMO)=27.41, P(PMO)<0.0001, n=4; post-hoc test *P<0.05, ***P<0.001 versus Ctrl PMO-injected group; #P<0.05 versus normal HTT. (D) CTG28 had no effect on the expression of control proteins ATXN2, ATXN3 and TBP. Student's t-test, n=4. (E) CTG28 injections decreased immobility time and increased latency to immobility and escape attempts of HdhQ7/Q150 knock-in HD mice in the tail suspension test. Two-way repeated-measure ANOVA; post-hoc test *P<0.05 versus Ctrl PMO-treated group; n=4.

FIG. 11 shows the transfection efficiency of PMO in vitro. HD patient-derived fibroblast cell line HD 10/109, neuroblastoma cell line SH-SYSY and mouse striatal cell line STHdh Q111/Q111 were transfected with 20 μM FITC-labeled Ctrl PMO for 48 hours. Ctrl PMO was found in the cytoplasm of all lines with a transfection efficiency of at least 90%. Green, fluorescein-labeled Ctrl PMO; Blue, 4′,6-diamidino-2-phenylindole (DAPI) as a nucleus marker. Scale bar, 20 μm.

FIGS. 12A-12G depict representative western blots for data presented in FIGS. 5A-5E and FIGS. 10F and 10G.

FIG. 13 shows that modification of DICER1 expression confirms that suppression of HTT expression by PMOs is not DICER-dependent. HD 19/109 cells were transfected with 100 pmol control (Ctrl) or DICER1 siRNAs, respectively, for 48 hours, and subsequently treated with 5 μM control or CTG22 PMO for another 48 hours. Levels of HTT and DICER1 were assessed by western blot (bottom). The ability of CTG22 to reduce HTT levels was not affected by the modulation of DICER1 level (top). Two-way ANOVA, F(Allele)=3.90, P(Allele)=0.0657, F(Treatment)=30.62, P(Treatment)<0.0001, n=3, post-hoc test NS=no significance versus Ctrl siRNA- and CTG22-treated groups. HTT antibody is shown in brackets. Experiment was performed three times.

FIG. 14 shows that the body weight of HdhQ7/Q150 mice is not changed after three ICV injections of CTG28 PMO. Body weight of HdhQ7/Q150 mice was measured once a week at indicated ages. Arrows show three ICV injections of Ctrl or CTG28 PMO. No loss of body weight was observed after injections of CTG28 PMO, compared with Ctrl PMO. Two-way repeated-measure ANOVA with Bonferroni's post-hoc test, F (age)=7.128, P (age)<0.0001, F (PMO)=0.0439, P (PMO)=0.8409; n=4.

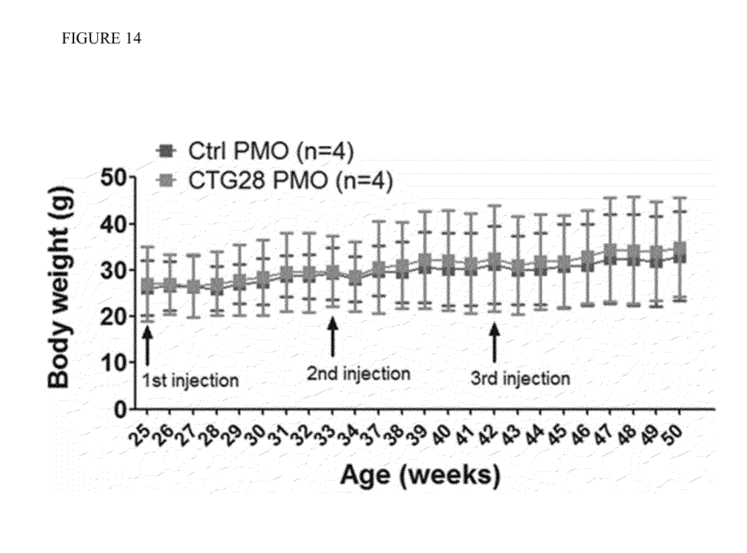

### DETAILED DESCRIPTION OF THE INVENTION

In accordance with an embodiment, the present invention provides an antisense phosphorodiamidate morpholino oligomer having the formula:

5′-(CTG)nC-3′ (I),

wherein n=5 to 15 trinucleotide repeats.

In accordance with another embodiment, the present invention provides an antisense phosphorodiamidate morpholino oligomer, wherein the antisense phosphorodiamidate morpholino oligomer is selected from the group consisting of:

(CTG22)
(SEQ ID NO: 1)
5′-CTGCTGCTGCTGCTGCTGCTGC-3′,
(CTG25)
(SEQ ID NO: 2)
5′-CTGCTGCTGCTGCTGCTGCTGCTGC-3′,
and
(CTG28)
(SEQ ID NO: 3)
5′-CTGCTGCTGCTGCTGCTGCTGCTGCTGC-3′.

Antisense phosphorodiamidate morpholino oligomers (hereinafter “PMO”) have been well studied as promising tools with potential to block ribonucleic acid transcription (Summerton, J; Weller, D (1997); Antisense & Nucleic Acid Drug Development 7 (3): 187-95), and as such have potential value as therapeutics whose purpose is to control protein expression.

As used herein, the terms “oligomer” and “oligonucleotide analog” may be used interchangeably with respect to the antisense PMO of the claimed subject matter. As used herein, the terms “antisense oligonucleotide analog” or “antisense compound” are used interchangeably and refer to a sequence of subunits, each having a pyrimidine or purine nucleobase carried on a backbone subunit composed of a morpholino group, and where the backbone groups are linked by substantially uncharged phosphorodiamidate groups that allow the bases in the compound to hybridize to a target sequence, such as, for example, in HTT mRNA by Watson-Crick base pairing, to form an RNA:PMO heteroduplex within the target sequence. The PMO may have substantially complete complementarity to the RNA target domain or near complementarity so that the degree of complementarity is in the range of about 80% to about 100%. PMO are designed to block or inhibit translation of the mRNA containing the target sequence.

The term “target sequence” refers to a portion of the target RNA against which the antisense agent is directed and will hybridize by Watson-Crick base pairing of an essentially complementary or nearly complementary sequence.

The term “knock down” refers to the inhibition or blocking of protein synthesis due to steric inhibition of the transcription process. In one aspect of the present invention, said knock down is a result of Watson-Crick pairing of an antisense PMO to RNA encoding the HTT protein or a mutant thereof, providing the prophylaxis or treatment of Huntington's disease caused by said mutant HTT protein.

The term “polynucleotide,” as used herein, includes and/or is synonymous with “nucleic acid,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

In accordance with an embodiment, the present invention provides an antisense phosphorodiamidate morpholino oligomer having a polynucleotide sequence of between 20 to 30 nucleotides in length, which is complimentary to the nucleotides immediately 3′ to the start codon of the HTT gene mRNA.

In accordance with a further embodiment, the present invention provides an antisense phosphorodiamidate morpholino oligomer having the sequence:

(HTTex1a)
(SEQ ID NO: 4)
5′-CCTTCATCAGCTTTTCCAGGGTCGC-3′.

In accordance with an embodiment, the present invention provides an antisense phosphorodiamidate morpholino oligomer having a polynucleotide sequence of between 20 to 30 nucleotides in length, which is complimentary to the nucleotides immediately 5′ from the start of the CAG repeat region of the HTT gene mRNA.

In accordance with another embodiment, the present invention provides an antisense phosphorodiamidate morpholino oligomer having the sequence:

(HTTex1b)
(SEQ ID NO: 5)
5′-GCTGCTGCTGCTGGAAGGACTTGAG-3′.

In accordance with an embodiment, the present invention provides a composition comprising at least one or more of the antisense phosphorodiamidate morpholino oligomers described herein.

The term “polyribonucleotide,” as used herein, includes “ribonucleic acid,” “oligoribonucleotide,” and “ribonucleic acid molecule,” and generally means a polymer of RNA which can be single-stranded or double-stranded, synthesized or obtained (e.g., isolated and/or purified) from natural sources, which can contain natural, non-natural or altered nucleotides, such as morpholinos, and which can contain a natural, non-natural or altered internucleotide linkage, such as a phosphoroamidate linkage or a phosphorothioate linkage, instead of the phosphodiester found between the nucleotides of an unmodified oligonucleotide. It may be suitable in some instances, in an embodiment, for the nucleic acids to comprise one or more insertions, deletions, inversions, and/or substitutions.

The nucleic acids can be constructed based on chemical synthesis and/or enzymatic ligation reactions using procedures known in the art. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The present invention also relates to compounds, compositions, and methods useful for modulating the expression and activity of a target gene of interest, or expression and/or activity by antisense PMOs of the present invention. As used herein, the instant invention features PMOs and methods used to modulate the expression of target genes of interest.

The term “modulate,” as used herein means that the expression of the target gene, or level of RNA molecule or equivalent RNA molecules encoding one or more target proteins or protein subunits, or activity of one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

The terms “inhibit,” “down-regulate,” “reduce,” or “knockdown,” as used herein, means that the expression of the target gene, or level of RNA molecules or equivalent RNA molecules encoding one or more target proteins or protein subunits, or activity of one or more target proteins or protein subunits, is reduced below that observed in the absence of the PMOs of the present invention. In an embodiment, inhibition, down-regulation or reduction with a PMO molecule is below that level observed in the presence of an inactive or attenuated molecule. In another embodiment, inhibition, down-regulation, or reduction with PMO molecules is below that level observed in the presence of, for example, a PMO molecule with scrambled sequence or with mismatches. In another embodiment, inhibition, down-regulation, or reduction of target gene expression with a nucleic acid molecule of the instant invention is greater in the presence of the nucleic acid molecule than in its absence.

In accordance with an embodiment of the present invention, the amount of time of exposure of the PMOs to the host cells, population of cells or subject should be sufficiently long to effect gene “knockdown” or modulation of the expression of the target gene in the host cell, population of cells or in the subject. The time for the desired effect varies with dosage, target, age and other factors known to those of skill in the art. Generally, the time of exposure of the PMOs to the host cells, population of cells or subject should range from about 1 hour to about 120 hours, preferably from about 1 hour to about 48 hours, more preferably from about 1 hour to about 24 hours.

In accordance with another embodiment, the present invention provides a method for reducing or inhibiting expression of HTT protein in a cell comprising contacting the cell with at least one or more of the antisense phosphorodiamidate morpholino oligomers described herein.

In accordance with still another embodiment the present invention provides a method for reducing or inhibiting expression of HTT protein in a subject comprising administering to the subject an effective amount of a pharmaceutical composition comprising at least one or more of the antisense phosphorodiamidate morpholino oligomers described herein.

By “gene”, or “target gene”, is meant, a nucleic acid that encodes a RNA, for example, nucleic acid sequences including, but not limited to, structural genes encoding a polypeptide. A gene or target gene can also encode a functional RNA (fRNA) or non-coding RNA (ncRNA), such as small temporal RNA (stRNA), miRNA, small nuclear RNA (snRNA), siRNA, small nucleolar RNA (snRNA), ribosomal RNA (rRNA), transfer RNA (tRNA) and precursor RNAs thereof

As used herein, the term “complementarity” or “complementary” means that a nucleic acid can form hydrogen bond(s) with another nucleic acid sequence by either traditional Watson-Crick or other non-traditional types. In reference to the polyribonucleotide molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively).

As used herein, the term “RNA” means a molecule comprising at least one ribonucleotide residue. By “ribonucleotide” is meant a nucleotide with a hydroxyl group at the 2′ position of a β-D-ribo-furanose moiety. The terms “RNA,” “ribonucleotides” and “polyribonucleotide,” also include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA, or internally, for example, at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

In accordance with the present invention, as used herein, the term “one or more PMO molecules” means that the PMOs of the present invention can comprise more than one PMO molecule. Furthermore, the more than one PMO molecules can include molecules having different nucleotide sequences directed to more than one mRNA nucleotide sequences. For example, in an embodiment, the present invention can comprise PMOs molecules having, two, three or four distinct nucleotide sequences specific for different target genes or different sequences of the same target gene.

The length of the PMO molecule can be any length greater than about 10 bp, which is capable of binding its complementary sequence on the mRNA of the target gene of interest in the cytosol of a cell or population of cells. The length of the PMO can be about 20 to about 30 bp, including, for example, 20 bp, 25 bp, and 30 bp.

It is contemplated that any of the PMO embodiments of the present invention described above can also encompass a pharmaceutical composition comprising the PMOs and a pharmaceutically acceptable carrier.

In accordance with another embodiment, the present invention provides a pharmaceutical composition comprising at least one or more of the antisense phosphorodiamidate morpholino oligomers described herein, and a pharmaceutically acceptable carrier.

With respect to nanoparticle compositions described herein, the carrier can be any of those conventionally used, and is limited only by physico-chemical considerations, such as solubility and lack of reactivity with the active compound(s), and by the route of administration. The carriers described herein, for example, vehicles, adjuvants, excipients, and diluents, are well-known to those skilled in the art and are readily available to the public. It is preferred that the carrier be one which is chemically inert to the active agent(s), and one which has little or no detrimental side effects or toxicity under the conditions of use. Examples of the carriers include soluble carriers such as known buffers which can be physiologically acceptable (e.g., phosphate buffer) as well as solid compositions such as solid-state carriers or latex beads.

The carriers or diluents used herein may be solid carriers or diluents for solid formulations, liquid carriers or diluents for liquid formulations, or mixtures thereof

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof

For liquid formulations, pharmaceutically acceptable carriers may be, for example, aqueous or non-aqueous solutions, or suspensions. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Intravenous vehicles include, for example, fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

The choice of carrier will be determined, in part, by the particular nanoparticle containing composition, as well as by the particular method used to administer the composition. Accordingly, there are a variety of suitable formulations of the pharmaceutical composition of the invention. The following formulations for parenteral, subcutaneous, intravenous, intramuscular, intraarterial, intrathecal and interperitoneal administration are exemplary, and are in no way limiting. More than one route can be used to administer the compositions of the present invention, and in certain instances, a particular route can provide a more immediate and more effective response than another route.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

As used herein the term “pharmaceutically active compound” or “biologically active compound” means a compound useful for the treatment or modulation of a disease or condition in a subject suffering therefrom. Examples of pharmaceutically active compounds can include any drugs known in the art for treatment of disease indications.

For purposes of the invention, the amount or dose of the PMOs of the present invention that is administered should be sufficient to effectively target the cell, or population of cells in vivo, such that the modulation of the expression of the target gene of interest can be detected, in the subject over a reasonable time frame. The dose will be determined by the efficacy of the particular PMO formulation and the location of the target population of cells in the subject, as well as the body weight of the subject to be treated.

The dose of the PMOs of the present invention also will be determined by the existence, nature and extent of any adverse side effects that might accompany the administration of a particular nanoparticle. Typically, an attending physician will decide the dosage of the PMO with which to treat each individual subject, taking into consideration a variety of factors, such as age, body weight, general health, diet, sex, compound to be administered, route of administration, and the severity of the condition being treated. By way of example, and not intending to limit the invention, the dose of the PMOs of the present invention can be about 0.001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight. In another embodiment, the dose of the PMOs of the present invention can be at a concentration from about 1 nM to about 10,000 nM, preferably from about 1 μM to about 50 μM.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of expression of HTT protein, or mutant HTT protein expression, or Huntington's disease in a mammal

As defined herein, in one or more embodiments, “administering” means that the one or more PMOs of the present invention are introduced into a sample having at least one cell, or population of cells, having a target gene of interest, and appropriate enzymes or reagents, in a test tube, flask, tissue culture, chip, array, plate, microplate, capillary, or the like, and incubated at a temperature and time sufficient to permit uptake of the at least one PMOs of the present invention into the cytosol, where it will bind to the mRNA of the target gene of interest and attenuate the expression of the target gene in the at least one cell or population of cells.

In another embodiment, the term “administering” means that at least one or more PMOs of the present invention are introduced into a subject, preferably a subject receiving treatment for a disease, and the at least one or more PMOs are allowed to come in contact with the one or more disease related cells or population of cells having the target gene of interest in vivo.

In accordance with another embodiment, the present invention provides a method for treatment of Huntington's disease in a subject comprising administering to the subject an effective amount of a pharmaceutical composition comprising at least one or more of the antisense phosphorodiamidate morpholino oligomers described herein.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In a further embodiment, the PMOs of the present invention can be used in combination with one or more additional therapeutically active agents which are known to be capable of treating conditions or diseases discussed above. For example, the described PMOs of the present invention could be used in combination with one or more known therapeutically active agents, to treat a disease or condition. Non-limiting examples of other therapeutically active agents that can be readily combined in a pharmaceutical composition with the PMOs of the present invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

In accordance with a further embodiment, the present invention provides a pharmaceutical composition comprising at least one or more of the antisense phosphorodiamidate morpholino oligomers described herein, at least one additional biologically active agent, and a pharmaceutically acceptable carrier.

### EXAMPLES

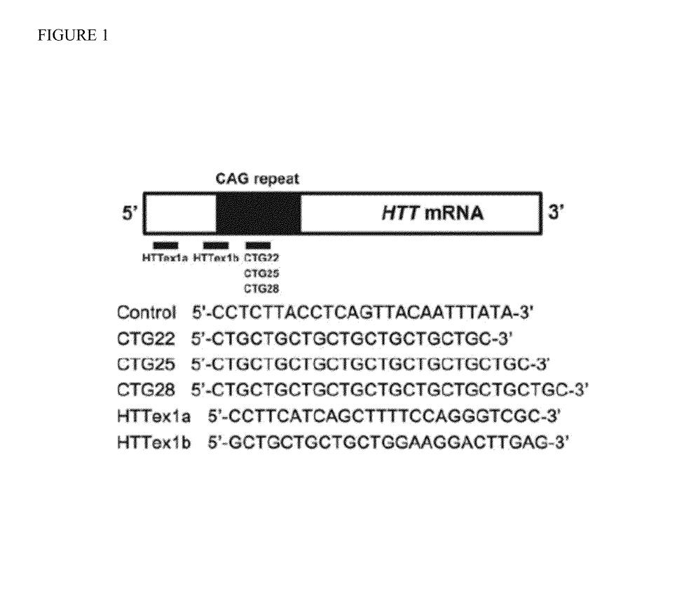

Reagents and antibodies. Standard control PMO, custom made PMOs and Endo-Porter (in DMSO solution) were obtained from Gene Tools, LLC (Philomath, Oreg.). The sequence for each of the PMOs used in this study is given in FIG. 1. Taqman Assay IDs for real-time PCR purchased from Applied Biosystems (Carlsbad, Calif.) were as follows: HTT, Hs00918174_m1; ATXN1, Hs00165656_m1; ATXN3, Hs01026440_g1; TBP, Hs00427620_m1; ATN1, Hs00157312_m1; GUSB, Hs00939627_m1; HTTASv1, described by Chung et al., Human Molecular Genetics, 20, 3467-3477 (2011).

For toxicity assay in cells, the Caspase-Glo 3/7 assay kit was used (G8091, Promega, Madison, Wis.). Control and DICER1 siRNAs were purchased from Ambion (Carlsbad, Calif.). The antibodies and concentrations used for immunoblotting and immunofluorescence were as follows: anti-huntingtin (MAB2166, 1:1000), anti-polyglutamine, 1C2 (MAB1574, 1:2000), anti-ataxin-3 (MAB5360, 1:500) anti-Androgen Receptor (06-680, 1:200), all from EMD Millipore (Billerica, Mass.); anti-TBP (sc-273, 1:200), anti-histone H1 (sc-8030, 1:200) and anti-Retinoic Acid Induced 1 (sc-365065, 1:100), all from Santa Cruz Biotechnology (Santa Cruz, Calif.); anti-β-actin (ab8224, 1:5000) from Abcam (Cambridge, Mass.); anti-DICER1 (SAB4200087, 1:200) from Sigma-Aldrich; anti-Rab7 (9367, 1:100) from Cell Signaling Technology (Danvers, Mass.). The secondary antibodies were Cy3-conjugated goat-anti-rabbit IgG, HRP-conjugated goat anti-rabbit IgG and HRP-conjugated goat anti-mouse IgG antibodies (Life Technologies, Grand Island, N.Y.).

Cell culture, transfection and PMO treatment. HD patient-derived fibroblast cell lines HD 18/44, HD 20/77 and HD 19/109 were obtained from the Baltimore Huntington's Disease Center. The fibroblasts were cultured in Minimal Essential Medium Eagle (MEM) supplemented with heat-inactivated 10% fetal bovine serum (FBS) and 1% MEM nonessential amino acid solution (NEAA). HEK293 cells (CRL-1573; ATCC, Manassas, Va.) and neuroblastoma cell line SH-SY5Y (CRL-2266, ATCC) were cultured in Dulbecco's Modified Eagle Medium (DMEM) with 4.5 g/L glucose and supplemented with 10% FBS. Mouse primary cortical neurons were prepared as previously described and cultured in Neurobasal Medium supplemented with 2% B27 supplements. All media and reagents used for cell culture were obtained from Life Technologies. All cells were maintained at 37° C. with 5% CO₂ and plated either in 6-well plates (100,000 cells/well) or in 96-well plates (5,000 cells/well) one day prior to siRNA transfection or PMO treatment. For knockdown of DICER1, 100 pmol of siRNA was transfected into HD 19/109 fibroblasts using Lipofectamine 2000 reagents, according to the manufacturer's manual (Life Technologies). For all PMO treatments, PMOs (at respective concentrations) and Endo-Porter (6 μM) were pre-mixed for 30 minutes and then added to the culture medium for 48 hours before collection for analysis.

Western blot. Following PMO treatment, cells were washed with PBS, and total protein was extracted using RIPA buffer (Sigma-Aldrich, St. Louis, Mo.) according to the manufacturer's protocol. For nuclear protein extraction, the NE-PER Nuclear Protein Extraction Kit (Pierce, Rockford, Ill.) was used according to the manufacturer's protocol. Total protein extracts from mouse brains were prepared following tissue homogenization in RIPA buffer. Protein samples were subjected to SDS-polyacrylamide gel electrophoresis (SDS-PAGE) using 3-8% tris-acetate gel (constant voltage of 120V, for immunoblotting of HTT) and 4-12% bis-tris gel (constant voltage of 100V, for immunoblotting of other proteins; Life Technologies). Proteins were transferred onto nitrocellulose membranes (Bio-Rad, Hercules, Calif.) at a constant voltage of 20V overnight and blocked with 5% milk in Tris-Buffered Saline containing 0.1% TWEEN 20 (TBS-T) for one hour at room temperature (RT). Membranes were incubated with primary antibodies overnight at 4° C. After washing with TBS-T, membranes were incubated with the corresponding secondary antibodies and protein bands were detected using the ECL Prime Western Blotting System (GE Healthcare, UK). Images were analyzed with ImageJ Software (NIH, rsbweb.nih.gov/ij/). Relative protein levels of TBP were obtained by normalizing TBP levels to that of histone H1, while β-actin was used as an internal control for other proteins.

RNA isolation and real-time PCR. Total RNA was isolated using TRIZOL reagent (Sigma-Aldrich), according to the manufacturer's manual. Total RNA was reverse-transcribed using random hexamer primers and SuperScript III reverse transcriptase (Life Technologies). The ABI7900HT detection system (Applied Biosystems) was used for real-time PCR. Gene-specific Taqman primers and probes (Applied Biosystems) were used to monitor the replication of PCR products. Human GUSB was selected as an internal control. Quantities of transcripts were obtained using the standard curve method and normalized to GUSB RNA levels. RNA expression levels were represented as ratio of the gene of interest to GUSB.

Caspase-3/7 activity assay. Cells were treated with PMOs in 96 well plates and incubated with 100 μL/well of Caspase-Glo 3/7 reagent for 1 hour at 37° C. Fluoroskan Ascent Microplate Fluorometer (Thermo Scientific, Waltham, Mass.) was used to quantify luminescence. Relative luminescence unit (RLU) measurements of all treated groups were normalized to that of the non-treated group.

LDH assay. Released LDH activity was measured according to the manufacturer's protocol (Roche, Berlin, Germany). Briefly, 50 μL of conditioned media from cell cultures were incubated with 50 μL of LDH assay buffer at room temperature for 20 minutes in the dark. The reaction mixture was stopped and the absorbance was read at 492 nm. Fresh medium was also assayed as background and subtracted from each group to get relative LDH activity. Relative LDH activity of the non-treated control group was defined as 100, and all other groups were normalized to this value.

Immunofluorescence. Cells were seeded onto Poly D-Lysine-coated cover slips in a 24-well plate at 1×10⁵cells/well and incubated with culture medium containing 20 μM fluorescein-labeled standard control PMO. Cells were then washed with PBS and fixed with 4% paraformaldehyde (PFA) in PBS for 15 minutes at room temperature. For endosome staining, HD 19/109 cells were permeabilized in PBS with 10% BSA and 0.1% Triton X-100 for 30 minutes and incubated in a primary antibody against Rab7 at 4° C. overnight, followed by washing and 1-hour incubation with a secondary antibody. All images were acquired using a Nikon Eclipse E400 epifluorescence microscope (Nikon, Tokyo, Japan) and MetaVue 4.6 software (Molecular Devices, Sunnyvale, Calif.).

ICV injection in HD mouse. The breeding and subsequent use of N171-82Q transgenic mice and Hdh^{Q7/Q150} knock-in mice were approved by the ACUC of Johns Hopkins University, Baltimore, Md. Mice were housed at the East Baltimore campus rodent vivarium and maintained on a standard circadian cycle with free access to water and standard chow.

Six-week-old N171-82Q mice were anesthetized via intraperitoneal injection of a mixture of 80 mg/kg ketamine and 1 mg/kg dexmedetomidine, diluted in 0.9% sterile saline. Mice were then immobilized in a stereotactic device and injected in the right lateral ventricle (1.0 mm posterior to Bregma, 1.3 mm right, 2.0 mm deep). 100 μg of previously freeze-dried PMO were resuspended in 2 μL saline to create a 6 mM injection solution, which was injected using a 2 μL Hamilton syringe over one minute. After injection, the needle was withdrawn and skin was closed with cyanoacrylate glue. Post-surgical health was monitored until sufficient recovery was attained in respiration and mobility levels. 14 days after ICV injection, mice were killed by rapid decapitation and frozen brain region punches were taken in a cryostat. Total lysate of the frontal cortex was prepared from each mouse and used in western blot analysis.

Six-month-old Hdh^{Q7/Q150} knock-in mice were subject to the same injection protocol for three injections at six, eighth and tenths of age. The mice's body weight was measured weekly from the first injection. Two months after the last ICV injection, the mice were examined in the tail suspension test and then killed. The ipsilateral hemisphere of each mouse brain was dissected into the frontal cortex, striatum and cerebellum for western blot analysis.

Tail suspension test. The tail suspension test is a standard mouse behavioral phenotyping procedure that is used to assess depressive-like phenotype: mice administered MAOIs and other pharmaceutical antidepressants display decreased immobility and increased latency to immobility. Mice were allowed to acclimatize to individual housing in an isolated behavioral suite for one day prior to testing. Each mouse was hung by the distal 2 cm of tail to a shelf positioned approximately 18 inches above the bench top. The mice were oriented such that the ventral aspect faced the observer. Each trial lasted 60 seconds and each mouse completed 5 trials separated by approximately 15 minutes. Each trial was recorded by a digital camera and later scored by 2 observers (one blinded) for time spent attempting escape, time spent immobile, and latency to immobility.

Statistical analysis. Experiments were repeated at least three times. Data were presented as mean±SD. The results were analyzed using Student's t-test, one-way analysis of variance (ANOVA) followed by Dunnett's post-hoc test, or two-way ANOVA followed by Bonferroni's post-hoc test as indicated. Statistical significance was set at P value<0.05.

### Example 1

The specificity and effectiveness of a PMO depend on its sequence and concentration, as well as on the length of the targeted CAG repeat.

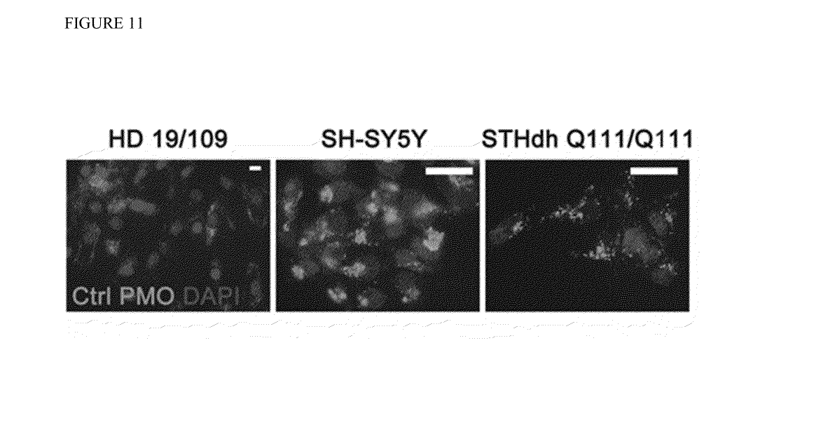

Based on the use of PMOs to ameliorate the toxicity of the CUG repeat expansion within the DMPK transcript associated with myotonic dystrophy 1 (DM1), we synthesized three CAG repeat-targeting PMOs, CTG22, CTG25 and CTG28, respectively (FIG. 1). The optimal length for a PMO is about 25 bases and the longest PMO that could be synthesized was 30 bases. The high transfection efficiency of approximately 90% was confirmed by delivering fluorescein isothiocyanate (FITC)-labeled standard control (Ctrl) PMO into multiple cell lines (FIG. 11) and visualizing FITC distribution through an eGFP filter. We evaluated PMO specificity and effectiveness in reducing mutant HTT protein levels in three HD patient-derived fibroblast cell lines, HD 18/44, HD 20/77 and HD 19/109 (Table 1; numbers indicate CAG repeat sizes within normal and mutant HTT alleles).

TABLE 1
CAG repeat sizes of fibroblast cell lines.

CAG repeat sizes of genes

| Cell line | HTT | ATXN2 | TBP | ATXN3 |
|---|---|---|---|---|
| HD 18/44 | 18/44 | 22/22 | 36/37 | 22/22 |
| HD 20/77 | 20/77 | 23/23 | 31/31 | 24/29 |
| HD 19/109 | 19/109 | 22/22 | 35/39 | 20/27 |

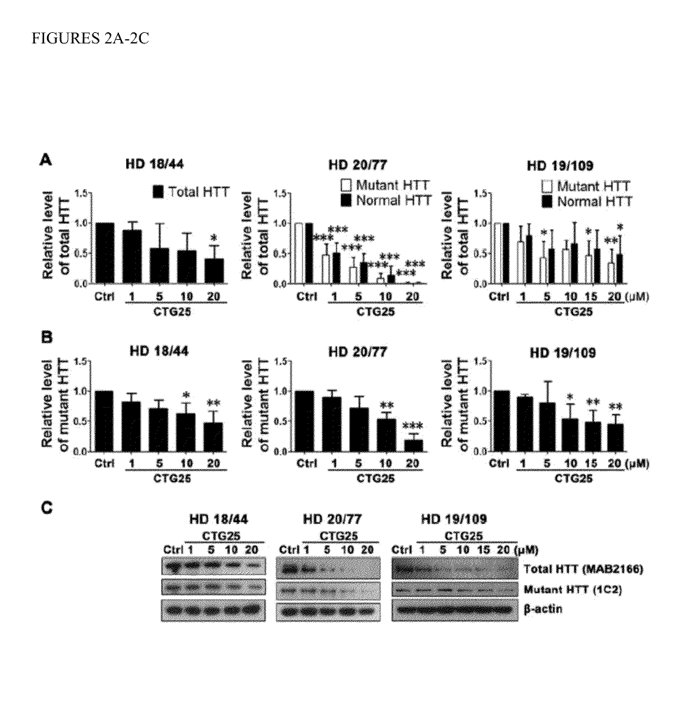

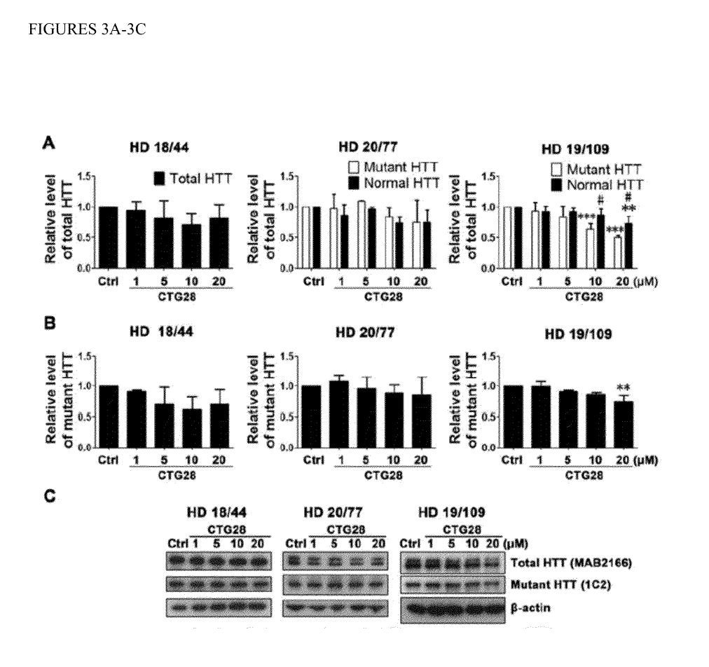

First, 1-20 μM of CTG25 PMO was applied to all three HD patient-derived fibroblast cell lines. 20 μM of a standard control PMO (Ctrl) served as a baseline. 48 hours after the treatment HTT protein levels were assessed by western blot. An antibody against full length HTT (MAB2166) was used to assess the total level of endogenous HTT in line HD 18/44, as allelic separation was not possible on the gel. The same antibody successfully resolved normal allele (lower band) from the mutant (upper band) in cell lines HD 20/77 and HD 19/109. In addition, the expression of mutant HTT was examined using 1C2 antibody. As shown in FIGS. 2A and C, CTG25 reduced total HTT expression in all three HD fibroblast lines. Blotting with 1C2 antibody demonstrated suppression of mutant HTT (FIGS. 2B and C). In cell lines HD 20/77 and HD 19/109 a non-significant trend for allelic selectivity of CTG25 was observed, and 10 and 20 μM reduced total HTT levels in the HD 18/44 line to approximately 40%, a potentially therapeutically meaningful level. We next hypothesized that increasing the CTG repeat length of the PMO may increase allelic selectivity due to increased cumulative binding of PMOs to the expanded CAG repeat. We observed that in cell line HD 19/109 treatment with 10 μM CTG28 reduced mutant HTT level to ˜65% of control levels, whereas normal HTT level remained at 90% of control. Increasing the concentration of CTG28 to 20 μM further decreased mutant HTT to ˜50% while normal HTT expression remained at 75% of control (FIGS. 3A and 3C). Specific staining for mutant HTT confirmed this finding (FIGS. 3B and 3C). However, this PMO did not significantly reduce mutant HTT protein in HD 18/44 or HD 20/77 (FIG. 3), indicating that increasing the length of the PMO CTG increases selectivity for long targeted repeats.

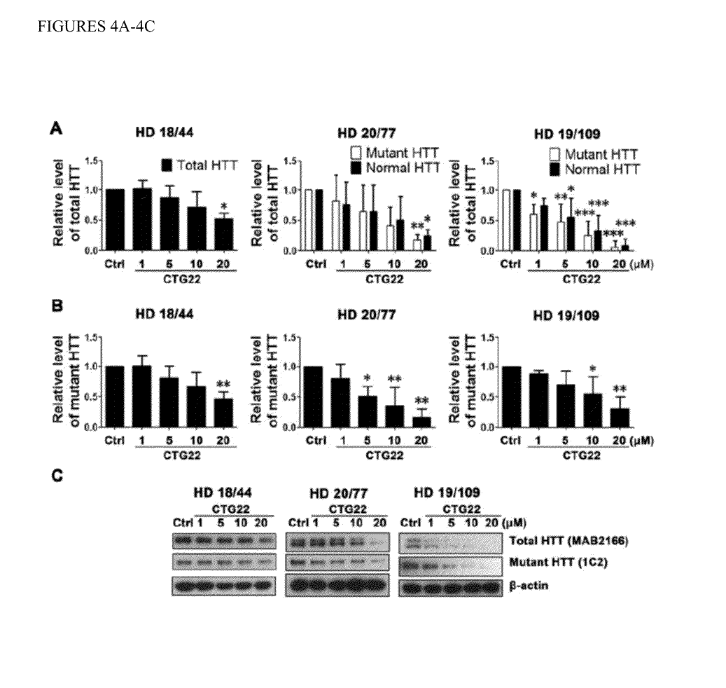

During the course of this study it was reported that a PMO consisting of 7 CTG triplets selectively decreased the expression of mutant HTT in HD-patient derived fibroblasts. We therefore examined the effect of a similar PMO, CTG22 (7 CTG triplets and an additional C) in more detail. We found that, like CTG25, total (FIGS. 4A and 4C) and mutant (FIGS. 4B and 4C) levels of HTT in all three HD cell lines were significantly reduced by CTG22 treatment (FIGS. 4A and 4C); however, no significant discrimination between normal and mutant HTT was observed in any of the lines. These results demonstrate that as CTG repeat length becomes shorter, HTT expression increases but allelic selectivity decreases. Taken together, we conclude that the allelic specificity and effectiveness of a repeat-targeting PMO depends on the length of the PMO repeat, the concentration of the PMO, and the length of the repeat in the targeted gene.

### Example 2

The off-target effect of CAG repeat-targeting PMOs depends on PMO sequence and concentration.

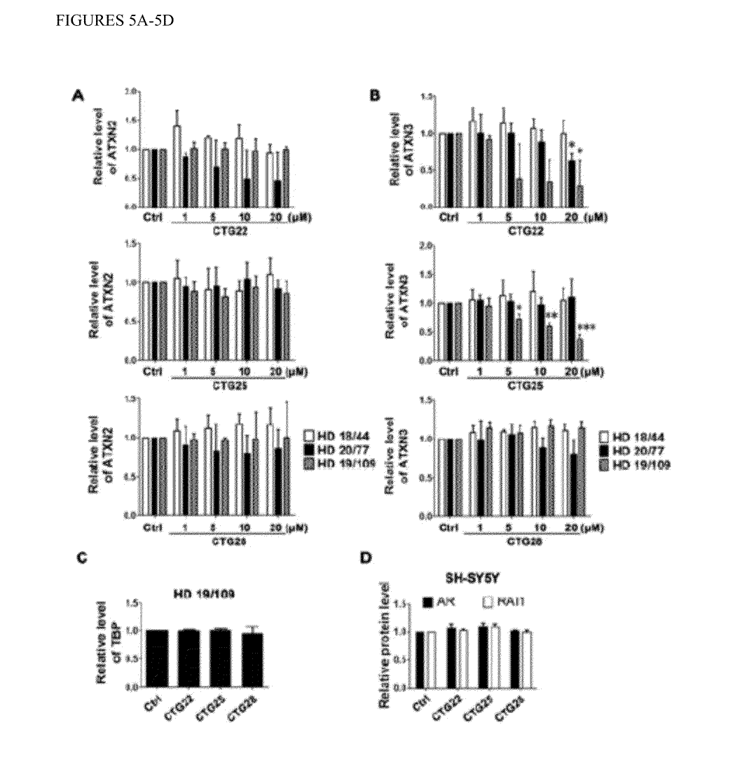

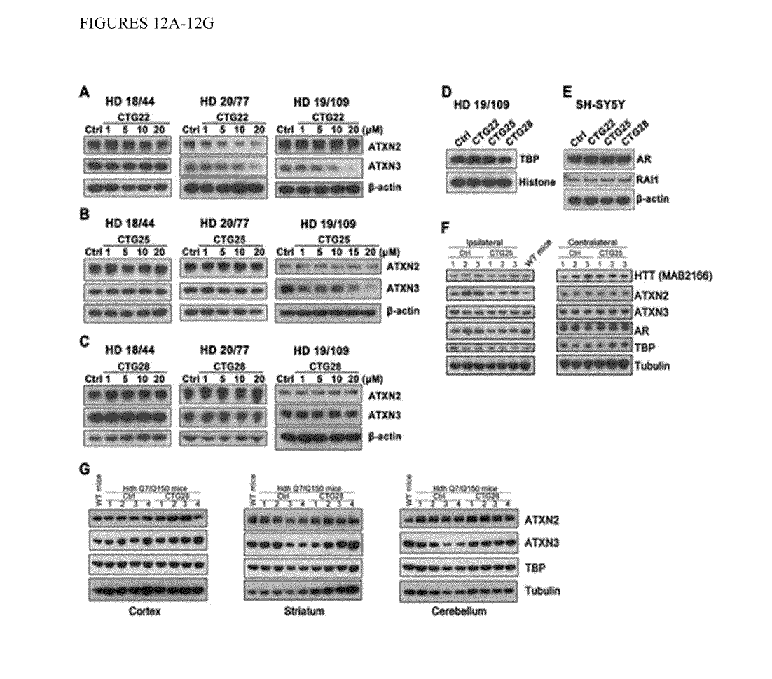

Off-target effect is an important criterion in evaluating the therapeutic potential of repeat-targeting ASO approaches. Besides HTT, there are at least 40 human genes with seven or more consecutive CAG triplets. To assess the off-target effect of repeat-targeting PMOs in this study, we chose five control genes that normally contain long CAG repeats: ataxin-2 (ATXN2), ataxin-3 (ATXN3), TATA box binding protein (TBP), androgen receptor (AR) and retinoic acid induced 1 (RAII). The repeat sizes of ATXN2, ATXN3 and TBP in three HD cell lines examined are shown in Table 1. Treatment with CTG22 significantly reduced the levels of endogenous ATXN3 in both HD 20/77 and HD 19/109 fibroblasts (FIG. 5B). Reduction of endogenous ATXN2 by PMO CTG22 in cell line HD 20/77 was not statistically significant (FIG. 5A). A high concentration of PMO CTG25 also inhibited endogenous ATXN3 expression to ˜40% of control in cell line HD 19/109 (FIG. 5B). PMO CTG28 had no inhibitory effect on endogenous ATXN2 and ATXN3 in any of the HD cell lines (FIGS. 5A and 5B). None of the three CAG repeat-targeting PMOs had an inhibitory effect on the expression of TBP in nuclear protein fraction extracts of cell line HD 19/109 (FIG. 5C). Since AR and RAH are expressed at low levels in HD patient-derived fibroblasts, we tested the effect of these CAG repeat-targeting PMOs on the endogenous levels of both proteins in the SH-SY5Y neuroblastoma cell line, and we observed no significant reduction (FIG. 5D). Representative western blot images for FIG. 5 are shown in FIG. 12.

The data suggest that the off-target effect of repeat-targeting PMOs on endogenous genes is dependent on the sequence of the PMO and possibly the sequence flanking the repeat and subsequent tertiary structure of each repeat locus, as the length of the repeat in the endogenous control genes examined is not directly correlated to the off-target effect of the PMO.

### Example 3

Non-CAG repeat-targeting PMOs reduce HTT levels with high target selectivity.

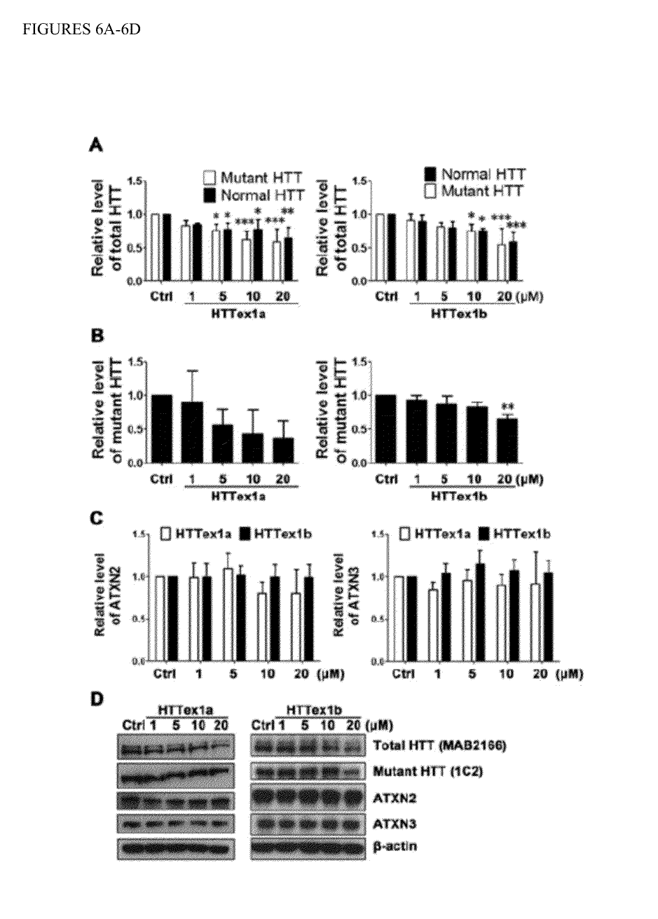

In addition to targeting the CAG repeat region in HTT RNA, targeting other regions can be a feasible therapeutic approach if levels of normal HTT remain sufficient to support normal cell function. We therefore designed two non-CAG repeat-targeting PMOs, HTTex1a and HTTex1b (FIG. 1), and examined their effectiveness in suppressing HTT expression in cell line HD 19/109. HTTex1a binds to the first 25 bases downstream of the start codon in HTT RNA, and HTTex1b binds to the region immediately upstream of the CAG repeat. As expected, HTTex1a and HTTex1b modestly decreased both normal and mutant HTT levels without allelic selectivity (FIGS. 6A, 6B and 6D). No effect on expression of endogenous ATXN2 or ATXN3 was detected with either of the PMOs (FIGS. 6C and 6D), as expected given the HTT specificity of these PMOs. The relatively small effect of these PMOs on HTT expression compared to repeat-targeting PMOs is presumably because only one PMO can bind to each transcript. Nonetheless, the comparative absence of off-target effects suggests the value of these PMOs.

### Example 4

RNA levels of HTT, HTTAS_v1 and control genes are not affected by PMOs.

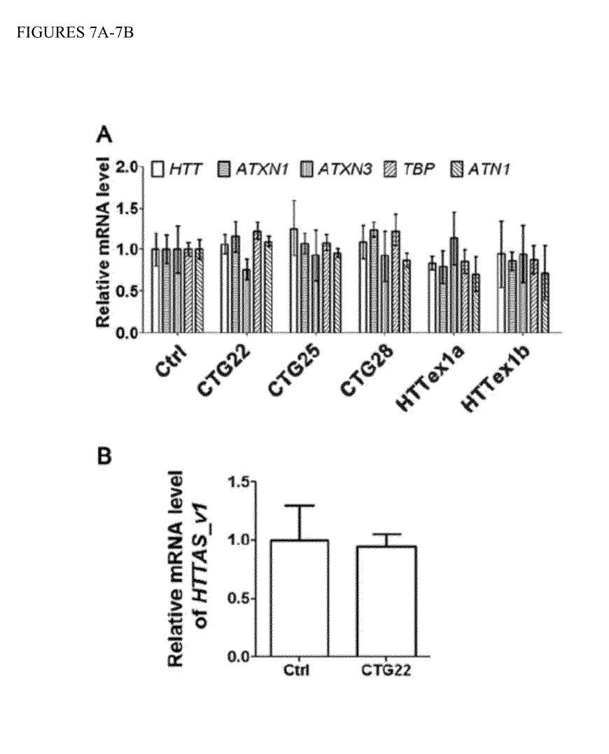

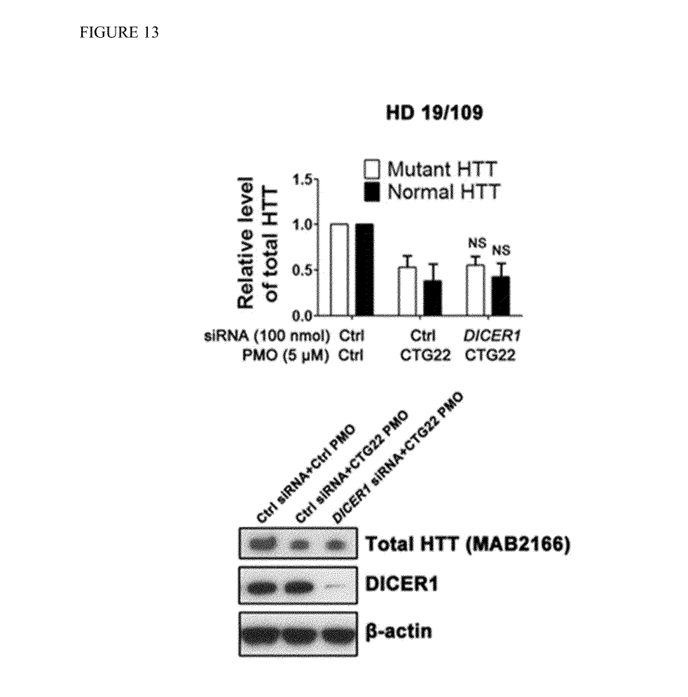

Since PMOs are designed to sterically block RNA translation through complementary binding with target RNA, PMO activity should not lead to degradation of targeted transcripts. To confirm this, we analyzed HTT RNA levels, as well as the RNA levels of four endogenous control genes including ataxin 1 (ATXN)1, ATXN3, TBP and atrophin-1 (ATN1) after treatment with each of the five PMOs. As expected, no significant effect on the levels of transcripts was observed (FIG. 7A). Since CTG22 was the most effective at reducing overall HTT levels (FIG. 4), we used this PMO to determine if PMO-HTT transcript hybridization affects levels of the antisense transcript at the HD locus, HTTAS_v1. Our results indicate that neither the levels of sense HTT nor HTTAS_v1 are significantly changed following introduction of the PMO (FIG. 7B). In addition, we observed that modulating the levels of the endoribonuclease dicer (DICER1) in HD cell line 19/109 transfected with PMO CTG22 has no significant effect on the levels of HTT (FIG. 13). Taken together, our experiments further confirm that PMO suppression of target expression does not involve degradation of either the targeted transcript or endogenous control genes.

### Example 5

PMOs show minimal cytotoxicity.

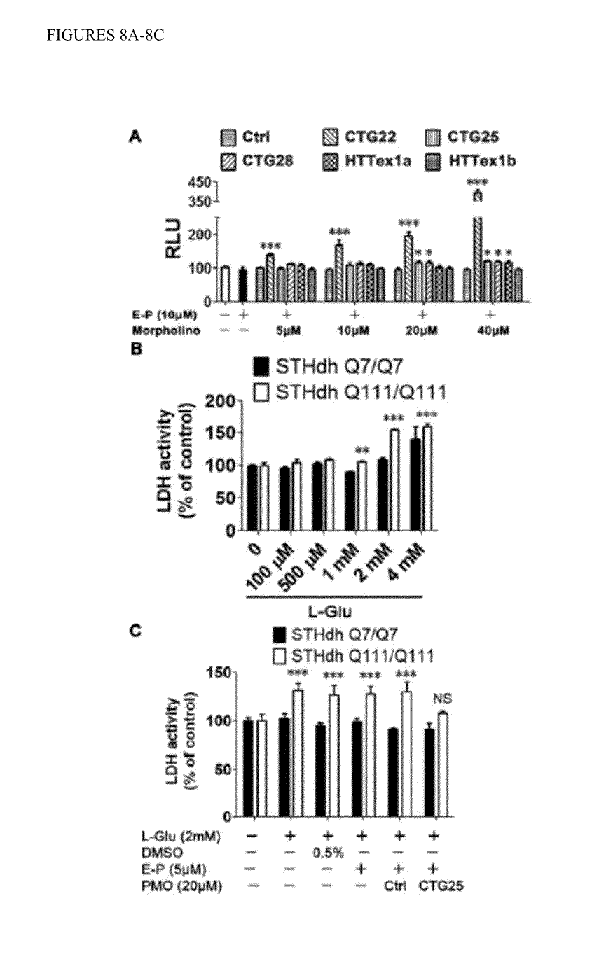

Any ASO-based strategy to lower HTT expression must be evaluated for non-specific cell toxicity. Unlike other ASOs, PMOs typically do not have cytotoxic effects. We confirmed that this general rule extended to our PMOs, as we were unable to detect toxicity in HD cell lines treated with a high concentration (20 μM) of each of the PMOs (data not shown). We next determined the toxicity of PMOs in normal HEK293 cells, as these cells tend to be more sensitive to toxins than fibroblasts. As shown in FIG. 8A, most of the PMOs were not toxic to HEK293 cells even at the highest concentrations, with the exception of CTG22, which was significantly toxic even at 5 μM. Since CTG22 induced the most off-target effect in HD cell lines (FIG. 5), we anticipate that this may reflect the capacity of PMOs with short repeats to target a number of endogenous genes containing CAG repeats. The data also demonstrates that off-target effects of PMOs, and hence potentially other ASOs, differ by cell type, and this phenomenon should be taken into consideration when evaluating ASOs as potential therapeutics.

### Example 6

PMOs protect cells from mutant HTT-induced toxicity.

Since CTG25 suppresses mutant HTT with minimal toxicity, we examined whether this PMO can protect neurons against mutant HTT-related toxicity. We used the STHdh cell model of HD, in which 5 mM sodium L-glutamate induces cytotoxicity in cell line STHdh Q111/Q111 expressing mutant HTT, but not in the control cell line STHdh Q7/Q7 expressing normal HTT. Using a modified protocol, we confirmed that incubation of 2 mM L-glutamate for 72 hours was toxic to STHdh Q111/Q111, but not to STHdh Q7/Q7 cells (FIG. 8B). We then observed that pre-treatment of STHdh Q111/Q111 cells for 48 hours with CTG25 protected the cells from glutamate-mediated mutant HTT-related neurotoxicity (FIG. 8C). This result indicates that PMO CTG25 can both suppress mutant HTT expression in patient fibroblasts and reduce HTT neurotoxicity in a cell model of HD.

### Example 7

A PMO's ability to suppress HTT in an allele-selective manner may be cell-type specific.

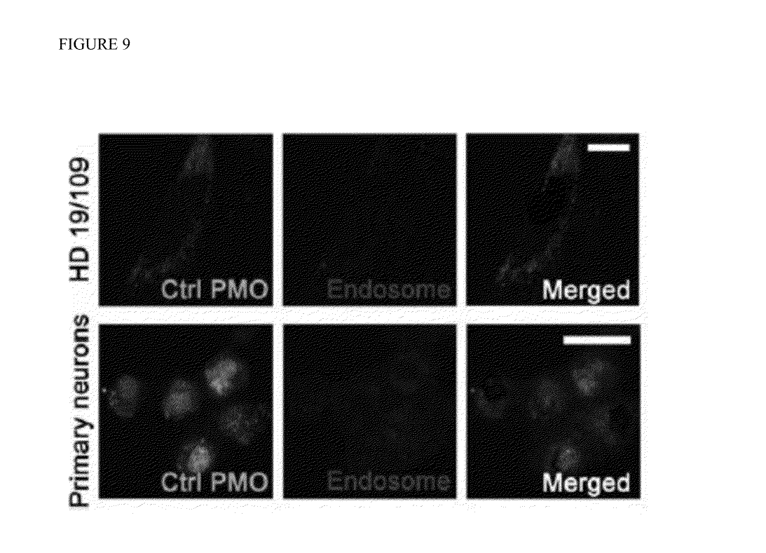

We next asked whether PMOs can suppress HTT expression in vivo. Since PMOs are transported into cells through endocytosis, and following release from the endosomes, freely diffuse into the cytoplasm and nucleus, we first examined to what extent PMOs can diffuse into cytoplasm and nucleus in neurons. We transfected a FITC-labeled control (Ctrl) PMO into mouse primary cortical neurons. Interestingly, we detected a much higher level of PMO release from neuronal endosomes than from fibroblast endosomes (FIG. 9). This finding confirms cell-type specific effect of PMOs, and provides a potential mechanistic explanation for cell-type specificity.

### Example 8

CTG25 and CTG28 inhibit mutant HTT expression in vivo.

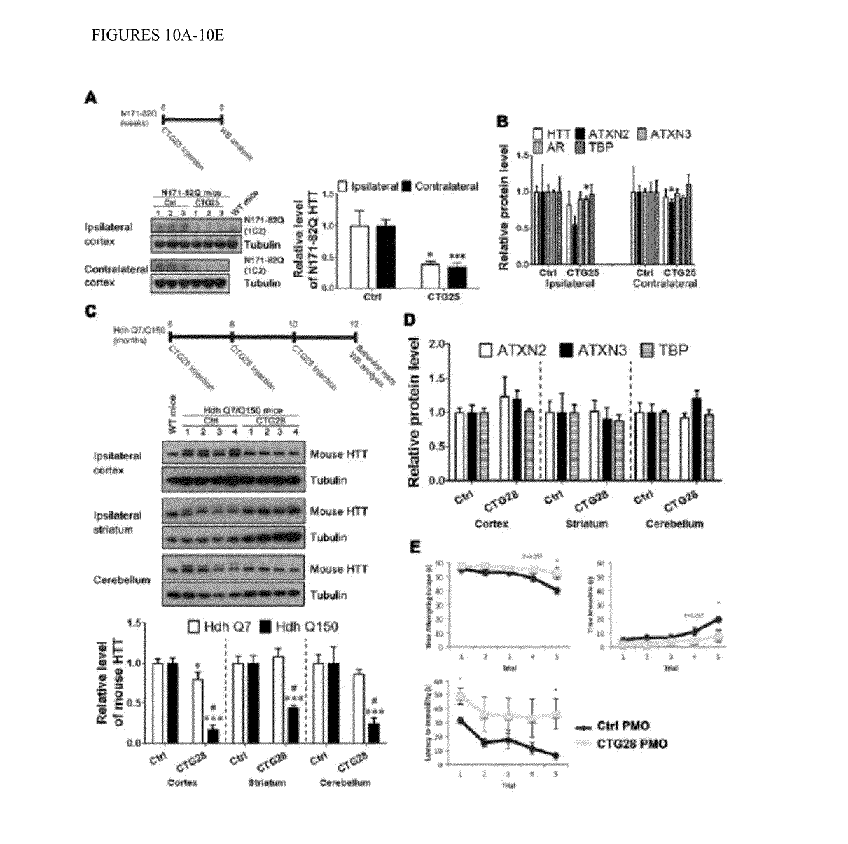

Since CTG25 reduced mutant HTT expression and rescued mutant HTT toxicity in vitro, we next examined the effect of CTG25 on HTT expression in vivo. We selected the N171-82Q transgenic HD mouse model for this experiment, as the repeat size in the model resembles that in HD 20/77 fibroblasts, in which PMO CTG25 was the most effective (FIG. 2A). A single, low-dose (100 μg) standard control (Ctrl) or CTG25 PMO was injected into the right lateral ventricle of six-week-old N171-82Q mice. The expression of the mutant HTT transgenic fragment in the frontal cortex was assessed two weeks post-injection. CTG25 was effective in reducing N171-82Q transgenic protein expression in the frontal cortex (FIG. 10A) without significant effect on the levels of endogenous normal HTT and control proteins containing polyglutamine repeats (FIG. 10B). The decrease of N171-82Q protein level in both ipsilateral and contralateral cortex suggests an efficient distribution of the PMO in vivo.

To further investigate the long-term efficacy and safety of PMOs, we also tested the effect of a repeat-targeting PMO in the HdhQ7/Q150 knock-in HD mouse model, which has both normal and mutant HTT expression at endogenous levels. We chose to use PMO CTG28 in this model based on its strong selectivity for expanded alleles in HD fibroblasts harboring 109 CAG repeats. Three successive, low-dose (100 μg) treatments of either Ctrl or CTG28 PMO were injected into the right lateral ventricle of six-month-old HdhQ7/Q150 knock-in mice at six, eight, and ten months of age. At the age of twelve months, mice were assessed in the tail suspension test, which has been used to measure depressive-like behavior of YAC128 HD mice, and the expression of HTT in various brain regions associated with the disease pathology was also assessed. No loss of body weight was observed in mice with CTG28 injections, suggesting that PMO CTG28 does not induce systemic deleterious effect (Fig. S4). CTG28 was effective in inhibiting the expression of mutant HTT (FIG. 10C, upper mouse HTT band) in the ipsilateral frontal cortex, ipsilateral striatum and cerebellum without affecting the levels of normal HTT (FIG. 10C, lower mouse HTT band) or control proteins containing polyglutamine repeats (FIG. 10D). These data suggest that CTG28 exhibits efficient distribution in the brain, good efficacy in reduction of mutant HTT, and little off-target effect in vivo. Representative western blot images for the data quantified in FIG. 10 are shown in FIG. 12. In addition, preliminary evidence also suggests that CTG28 improves the behavioral phenotype of HdhQ7/Q150 HD mice (FIG. 10E). Mice injected with CTG28 showed decreased immobility, increased latency to immobility and increased escape attempts compared to mice with Ctrl injections in the tail suspension test, an assay generally interpreted as a test of depressive-like phenotype.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An antisense phosphorodiamidate morpholino oligomer having the formula:
5′-(CTG)ₙC-3′ (I),
wherein n=5 to 15 trinucleotide repeats.

2. The oligomer of claim 1, wherein n=7 to 9.

3. The oligomer of claim 1, wherein the antisense phosphorodiamidate morpholino oligomer is selected from the group consisting of:
(CTG22)
(SEQ ID NO: 1)
5′-CTGCTGCTGCTGCTGCTGCTGC-3′,
(CTG25)
(SEQ ID NO: 2)
5′-CTGCTGCTGCTGCTGCTGCTGCTGC-3′,
and
(CTG28)
(SEQ ID NO: 3)
5′-CTGCTGCTGCTGCTGCTGCTGCTGCTGC-3′.

4. An antisense phosphorodiamidate morpholino oligomer having a polynucleotide sequence of between 20 to 30 nucleotides in length, which is complimentary to the nucleotides immediately 3′ to the start codon of the HTT gene mRNA.

5. The oligomer of claim 4, having the sequence:
(HTTex1a)
(SEQ ID NO: 4)
5′-CCTTCATCAGCTTTTCCAGGGTCGC-3′.

6. An antisense phosphorodiamidate morpholino oligomer having a polynucleotide sequence of between 20 to 30 nucleotides in length, which is complimentary to the nucleotides immediately 5′ from the start of the CAG repeat region of the HTT gene mRNA.

7. The oligomer of claim 6, having the sequence:
(HTTex1b)
(SEQ ID NO: 5)
5′-GCTGCTGCTGCTGGAAGGACTTGAG-3′.

8. A composition comprising at least one or more oligomers of claim 1.

9. A pharmaceutical composition comprising at least one or more oligomers of claim 1 and a pharmaceutically acceptable carrier.

10. The composition of claim 8, further comprising at least one additional biologically active agent.

11. A method for reducing or inhibiting expression of HTT protein in a cell comprising contacting the cell with at least one or more oligomers of claim 1.

12. A method for reducing or inhibiting expression of HTT protein in a subject comprising administering to the subject an effective amount of the composition of claim 9.

13. The method of claim 12, wherein the oligomer is CTG28.

14. A method for treatment of Huntington's Disease in a subject comprising administering to the subject an effective amount of the composition of claim 9.
